(19) United States
(12) Patent Application Publication
(10) Pub. No.: US 2012/0244178 A1
(43) Pub. Date: Sep. 27, 2012

(54) Plasmodium falciparum antigens

(76) Inventors: Denise Doolan, QLD (AU); Angela Trieu, QLD (AU); Phillip L. Felgner, Rancho Santa Fe, CA (US)

(21) Appl. No.: 13/426,768

(22) Filed: Mar. 22, 2012

Related U.S. Application Data
(60) Provisional application No. 61/467,517, filed on Mar. 25, 2011.

(51) Int. Cl. A61K 39/015; A61P 33/06; A61P 37/04; C07K 14/445; C12N 15/63

(52) U.S. Cl. 424/191.1; 530/350; 435/320.1

(57) ABSTRACT

The invention relates to antigens, associated with sterile immunity, and methods of their use, in an immunogenic formulation to confer an immune response against Plasmodium falciparum. The inventive antigens were identified by their association with sterile immunity against malaria.

This application claims the benefit of U.S. Provisional Application No. 61/467,517, filed Mar. 25, 2011, which is incorporated herein by reference.

### BACKGROUND OF INVENTION

1. Field of Invention

The inventive subject matter relates to DNA sequences and polypeptides from Plasmodium falciparum for use as an anti-malaria vaccine component and methods of inducing an immune response to these antigens.

2. Background Art

Malaria is caused by the vector borne organism Plasmodium spp. The parasite has a complex lifecycle requiring stage specific expression of proteins. These proteins can be expressed at different stages or be specific to stages. Malaria is an extremely important disease, with over 3 billion people living in malaria endemic areas. Over 1 million deaths are attributable to malaria per year. The emergence of drug resistant strains has compounded the problem of treating the disease. Unfortunately, no FDA-approved vaccine exists.

The entire genomic sequence of P. falciparum has been sequenced (Bowman et al., Nature, 400: 532-538 (1999). Gardner, et al. Nature, 419: 498-511 (2002)). The rodent malaria parasite, P. yoelii has also been sequenced (Carbon et al., Nature, 419: 512-519 (2002)). Despite this, however, the development of efficacious anti-malaria vaccines has been severely hampered by the paucity of promising antigens. Sequencing of the Plasmodium falciparum and Plasmodium yoelii genomes yielding identification over 5,200 genes in the genome. However, despite the large number of potential gene targets, use of the data set alone will not likely result in new vaccine constructs. Consequently, only 0.2% of the P. falciparum proteome is undergoing clinical testing. Moreover, these vaccine candidate antigens have failed to induce significant protection in volunteers. Nevertheless, immunization of mice and humans with radiation-attenuated sporozoites results in a high-grade immunity (>90%), suggesting that development of effective anti-malaria vaccines are possible. This protective immunity appears to target multiple sporozoite and liver stage antigens.

### SUMMARY OF THE INVENTION

The invention relates to a vaccine composition and method of immunizing against Plasmodium falciparum. The inventive composition comprises P. falciparum liver-stage proteins associated with sterile protection against infectious P. falciparum. In one embodiment, the proteins can be incorporated into an immunogenic composition, singly or in multiple combinations, as subunit antigens. In another embodiment, for maximal immunogenicity, all identified proteins are included in a single immunogenic composition. Alternatively, multiple combinations of the proteins are administered in an immunization regimen through more than one immunogenic composition, each combination containing a specific combination of said immunogenic proteins. In one embodiment, the immunogenic composition comprises one of the identified proteins that has been isolated and purified. In another embodiment, the immunogenic composition comprises two or more, and up to all 19, of the identified proteins that have been purified and isolated.

In another embodiment, DNA encoding one or more of these proteins can be incorporated into vectors suitable for in vivo expression in a mammalian host. The expressed and purified proteins can then be administered, in one or multiple doses, to a mammal, such as humans. In this embodiment, DNA encoding one or more of the sterile-immunity associated proteins can be inserted into suitable expression systems. Suitable expression systems include, but are not limited to, adenoviral based systems, such as in Bruder, et al (patent application publication number US 20080248060, published Oct. 9, 2008) or a DNA plasmid system. In this embodiment, other vector systems include the DNA encoding P. falciparum is administered as an insert of the suitable expression system and expressed in vivo. In this embodiment, an immunogenic composition can comprise DNA encoding one or more, or all, of the sterile-immune associated proteins. The proteins can be expressed by a single vector encoding one or more of the proteins or by multiple expression systems suitable for expression of DNA in a mammal.

### BRIEF DESCRIPTION OF DRAWINGS

FIG. 1. Summary of selection criteria and AUC profile. (A) Analyses were conducted to identify antigens that are associated with sporozoite-induced protection. After spot quantification, the signal intensity was asinh/vsn log transformed to control variance, scaled by the negative control, post-immunization (post-I) and pre-challenge (pre-C) time points combined, and then analyzed according to defined statistical (Bayes-regularized t-tests, area under the receiver operating characteristics curve (AUC) analysis) and biological criteria. A total of 86 fragments (78 P. falciparum proteins) were identified by either approach through the global irradiated sporozoite list. Sterile immunity-associated proteins were in common to both approaches. (B) AUC values of all antigens for the not protected and protected cohorts were determined by R statistical environment software (available through: www.r-project.org). Antigen rank is plotted relative to their AUC value. An AUC value approaching 1.0 suggests a very strong association of an antigen to protection induced by irradiated sporozoite immunization; an AUC value of 0.5 indicates pure chance. An AUC value of 0.7 was chosen as a threshold for positivity.

FIG. 2. Magnitude and frequency of recognition of the proteins in Table 1 associated with irradiated sporozoite induced protection. For the antigens (ranked by AUC), the cumulative signal intensity representing the sum of signal intensities for each antigen by all subjects from protected or not protected groups are presented. ***P<0.0055.

FIG. 3. Magnitude and frequency of recognition of the proteins in Table 1 associated with irradiated sporozoite induced protection. Frequency of recognition of proteins by protected (P) and not protected (NP) individuals. # represents current clinical candidates: AMA1 (PF11_{—}0344); CSP (PFC0210c); SSP2/TRAP (PF13_{—}0201).

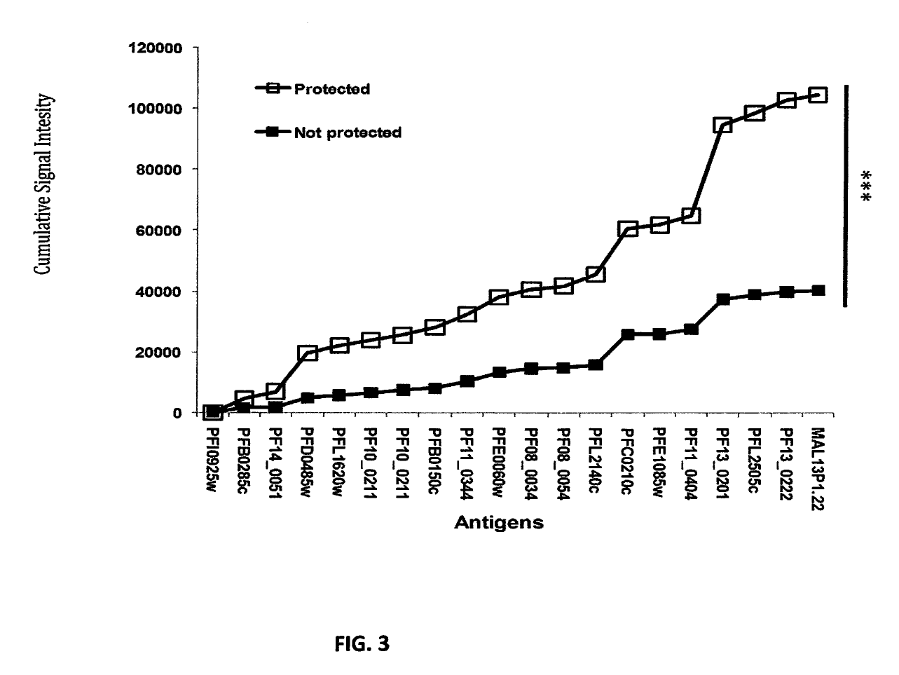

FIG. 4. Magnitude and frequency of recognition of the proteins in Table 1 associated with irradiated sporozoite induced protection. Average signal intensities of the antigens for each clinical group (protected, white bar; not protected, black bar) are presented as histograms, with antigen IDs listed on the x-axis. The average signal intensity (±s.e.m) for each antigen is shown. Antigens are ordered by decreasing AUC value.

### DETAILED DESCRIPTION OF PREFERRED EMBODIMENTS

As used, herein, “sterile immunity” refers to immunity, whereby the causative agent of the targeted disease causing organism is eliminated or inhibited from causing disease.

As used herein, the term “polypeptide” refers to a polymer of amino acids and does not refer to a specific length of the product. Proteins are included within the definition of polypeptides. As used herein, the proteins may be prepared for inclusion of an effective amount of one or more polypeptides described herein into an immunogenic composition by a number of means. For example, they may be included by first expressing the appropriate gene fragments by molecular methods, expression from plasmids or other expression systems such as viral systems and then isolated.

As used herein, “immunogenic fragments” of proteins refers to regions of proteins at least 8 amino acids in length, with the amino acid sequence derived from said protein, the fragment being capable of inducing an immune response or that is recognized by immune cells.

As used herein, “derivatives” of a protein is where a protein has more than 80% amino acid sequence identity to the sequences described herein. In this context, the term “identity” refers to two or more sequences or subsequences that are the same or have a specified percentage of amino acid residues that are the same, when aligned for maximum correspondence. Where sequences differ in conservative substitutions, i.e., substitution of residues with identical properties, the percent sequence identity may be adjusted upwards to correct for the conservative nature of the substitution.

As used herein, an aspect of the invention relates to nucleotide sequences that encode all or a substantial portion of the amino acid sequence encoding identified proteins or substantial portions, thereof. A “substantial portion” of a protein comprises enough of the amino acid sequence to afford putative identification of the protein the sequence encodes (Altschul, et al., J. Mol. Biol. 215: 403-410 (1993)). Furthermore, in general, this is approximately nine or more contiguous amino acids can lead to identification of the protein as homologous to a known protein. “Orthologous” nucleotide sequences are sequences encoding for proteins of the same function but in different species.

“Antigen” is a chemical moiety containing at least one epitope capable of stimulating or reacting with immune products, such as antibody or T-cells. An “immunogenic composition” refers to a chemical, compound or formulation that, once administered, will elicit an immune response; A “vaccine” is an immunogenic composition used to induce protective immunity; A “DNA expression system” is a molecular system containing plasmid or closed loop DNA containing elements for expressing an inserted DNA sequence as polypeptide; A “viral expression system” is any viral based system, including viral-like particles or viral replicons, containing elements for expressing an inserted DNA sequence as a polypeptide.

### Example 1

### Identification of Sterilely-Immune Associated Proteins

This example illustrates the identification of proteins associated with sterile immune against malaria. In this example, volunteers, experimentally immunized with radiation attenuated P. falciparum sporozoites (Irrspz), were clinically divergent after challenge with infectious P. falciparum sporozoites; six individuals were sterilely-protected and were classified as sporozoite-immune (IrrSpz protected) and five individuals developed blood stage parasitemia and were classified as sporozoite-exposed but non-immune (IrrSpz not protected).

Subjects were experimentally immunized with radiation-attenuated P. falciparum, (Pf(3D7)), sporozoites and challenged with P. falciparum-infected Anopheline mosquitoes, as described previously (Hoffman, et al., J. Infect. Dis. 185:1155-1164 (2002); Egan, et al., Am. J. Trop. Med. Hyg. 49:166-173 (1993)). Subjects were monitored daily, post-challenge, by thin blood smears to determine if they developed blood stage malaria. A complete absence of blood stage parasitemia during the 28 day follow up was considered sterile protection.

Six sporozoite-immunized volunteers were protected against sporozoite challenge and five were not protected (i.e. developed clinical malaria). One individual is represented in both groups since he was not protected in the initial challenge but was after a second series of immunizations. Plasma was collected from each individual before immunization (pre-immunization), post third immunization, at the completion of the immunization series (5th, 6th, or 7th immunization), immediately prior to challenge (pre-challenge) and following challenge (post-challenge). An infectivity control group (n=3) was simultaneously infected with the same P. falciparum-infected mosquitoes used for challenge to demonstrate parasite infectivity; plasma was collected from these individuals at corresponding time points before (pre-challenge) and after (post-challenge) challenge. An additional group (n=5) were mock immunized by the bite of non-infected mosquitoes and plasma was collected at the time points corresponding to pre-immunization, post-third immunization and post last immunization time points of the IrrSpz immunized subjects. Plasma collected from volunteers with no known history of malaria exposure (n=10) was also evaluated.

To identify antigens associated with IrrSpz-induced protection, plasma collected from protected, not protected, infectivity, mock immunized and naïve individuals at different stages of the immunization process (or corresponding time points for the non-immunized individuals) were probed on P. falciparum microarrays against 2,320 fragments, representing 1,200 P. falciparum proteins. Antibody recognition of each fragment was then assessed.

There were distinct antibody profiles for each immunization group, with variability in responses between individuals within all groups. Overall, a markedly different pattern in antibody recognition was apparent between the protected and not protected groups, consistent with previous data (Doolan, et al, Proteomics 8:4680-4694). There was no difference in antibody reactivity at pre-challenge and post-challenge time points for protected individuals who did not develop blood stage parasitemia or clinical disease (P<0.6) and no change in the number of antigens recognized (340 pre-challenge vs. 380 post-challenge).

Putative proteins, and protein and DNA sequences, were derived from the P. falciparum genomic sequence database (www.plasmodb.org) and selected based on stage-specific transcription or protein expression, subcellular localization, secondary protein structure, and documented immunogenicity in humans or animal models; this list included all putative P. falciparum proteins with evidence of expression at some point during the parasite life cycle by MudPIT (multidimensional protein identification technology) (Florens, et al., Nature 419:520-526 (2002)), www.plasmoDB.org) at the time of antigen selection (n=1049).

Selected genes were amplified by polymerase chain reaction (PCR) amplification of P. falciparum genomic DNA (3D7 strain) using custom PCR primers that included homologous cloning sites to the pXT7 plasmid (Davies, et al., Proc. Natl. Acad. Sci. (USA) 102:547-552 (2005)). Due to restrictions in producing long PCR products, proteins with exons longer than 3000 bp were divided into multiple overlapping sections, with 50 nucleotide overlaps. PCR reactions were carried out in a 50 μl reaction volume containing 1-10 ng of P. falciparum genomic DNA (gDNA) (3D7 strain), 0.04 U/μl proofreading Taq polymerase (Triplemaster™, Eppendorf, Hauppauge, N.Y.), and 0.4 mM each dNTPs, with the following cycling conditions: 95° C. for 3 min; 35 cycles of 95° C. for 15 s, 40° C. for 30 s and 50° C. for 60 s/kb; and a final extension of 50° C. for 10 min. For some proteins that proved difficult to amplify, 50 ng of gDNA was used. Products were visualized by agarose gel electrophoresis, and quantified by fluorometry (Picogreen™, Molecular Probes®, Invitrogen, Carlsbad, Calif.).

Plasmids were created from the PCR amplified fragments using in vitro recombination cloning and the pXT7 cloning vector, which encodes an N-terminal 10× Histidine (H is) and C-terminal Haemagglutinin (HA) tag (3.2 kb, KanR). Briefly, 1 ng of Bam HI, digested, linearized pXT7 template and custom primers 5′-CTACCCATACGATGTTCCGGATTAC and 5′-CTCGAGCATATGCTTGTCGTCGTCG were used to generate a linear acceptor vector containing the target gene by PCR (50 μl reaction) with 0.02 U/μl Taq polymerase, 0.1 mg/ml gelatine (Porcine), and 0.2 mM each dNTPs. The following cycling conditions were used: 95° C. for 5 min; 30 cycles of 95° C. for 0.5 min, 50° C. for 0.5 min and 72° C. for 3.5 min; and a final extension of 72° C. for 10 min. After purification (Qiagen, Valencia, Calif.), PCR products were visualized by gel electrophoresis, and quantified by fluorometry (Picogreen™, Molecular Probes®, Invitrogen, Carlsbad, Calif.).

Open reading frames (ORFs) were cloned into the linearized pXT7 plasmid by a recombination reaction as previously described (Davies, et al., Proc. Natl. Acad. Sci. (USA) 102:547-552 (2005). Briefly, a 20 μl mixture of linear vector and PCR-generated ORF fragment at a 1:1 molar ratio between vector and insert was transformed into DH5a competent cells without further purification and incubated for 1 h at 37° C., before dilution into an overnight culture of 3 ml LB broth containing Kanamycin 50 μg/ml. Plasmids were isolated and purified using the QIAprep™ spin miniprep, (Qiagen, Valencia, Calif.) without further selection. A subset of these plasmids was sequence confirmed.

Protein expression and detection was conducted using E. coli in vitro cell-free transcription and translation reactions (rapid translation system (RTS) 100 E. coli HY kits, Roche). Reactions were carried out in 25 μl volumes with a 5-hour incubation at 30° C., according to manufacturer's instructions. For quality control purposes, relative protein expression efficiency for approximately 31% of all ORFs was assessed by immunodot blots by spotting 0.3 μl of the RTS reaction on nitrocellulose (NC) and air drying before blocking in 5% nonfat milk powder in TBS containing 0.05% polysorbate 20. Dot blots were stained with mouse anti-polyHlS mAb (clone HIS-1; Sigma) and rat anti-HA mAb (clone 3F10) (F. Hoffmann-La Roche Ltd, Basel, Switzerland) and detected with alkaline phosphatase-conjugated goat anti-mouse IgG (H+L) or goat anti-rat IgG (H+L) secondary antibodies respectively or with human hyperimmune plasma (diluted 1:1000 in blocking buffer with 10% E. coli lysate) followed by alkaline phosphatase-conjugated goat anti-human IgG secondary antibody (H+L). Blots were visualized with nitroblue tetrazolium (NBT) developer.

Microchips were prepared by mixing 15 μl of RTS reaction with 10 μl of 0.125% polysorbate 20/PBS, and then 15 μl volumes were transferred to 384 well plates. Plates were centrifuged (1600×g, 5 min) to pellet any precipitates and proteins in the supernatant were immediately printed without further purification onto 3-pad NC-coated FAST™ glass slides (Schleicher & Schuell, Bioscience, Inc., Keene, N.H.) using an OmniGrid 100 microarray printer (Genomic Solutions, Ann Arbour, Mich.). Arrays were allowed to dry and stored away from light at room temperature in a desiccator.

RTS reactions carried out in the absence of DNA plasmids were printed on each array as negative or non-differentially recognized control spots. Purified human total IgG and Epstein-Barr nuclear antigen 1 (EBNA1) protein were also printed in serially diluted concentrations on each array, as probing and plasma controls respectively.

Since high titers of anti-E. coli antibodies present in the human plasma could mask protein-specific reactivity in the arrays, plasma were pre-absorbed against E. coli lysate in protein array blocking buffer (Schleicher & Schuell) (1:100 dilution) for 30 minutes at room temperature (Davis, et al., Proc. Natl. Acad. Sci. (USA) 102:547-552 (2005).

Slides were rehydrated and blocked in blocking buffer for 30 min at room temperature. Then 500 μl plasma diluted 1:100 in blocking buffer was added to each pad and slides were incubated overnight at 4° C. with a gentle constant speed on a platform rocker (Ratek™, InVitro Technologies). Serum antibodies were detected with biotin-conjugated goat anti-human IgG secondary antibody (1:1000 dilution, 1 h at room temperature with gentle constant speed, Fc fragment, (Jackson ImmunoResearch Laboratories, Inc, West Grove, Pa.) and visualized with a streptavidin P-3-conjugated antibody (1:200 dilution, 1 h at room temperature with gentle constant speed. Air dried slides were scanned on an Axon GenePix™ 4300A array scanner (Molecular Devices, CA) and fluorescence intensities quantified using the Axon GenePix Pro 7™ software (Molecular Devices, Sunnyvale, Calif.). Using the above software, all signal intensities were corrected for spot-specific background, where the background value for a spot was calculated from a region surrounding the spot.

To validate the P. falciparum protein microarrays, antibody reactivity to three well characterized malaria vaccine candidates, CSP, AMA1 and merozoite surface protein 2 (MSP2), were expressed via the RTS system or using traditional methods and were printed on the same protein microarray chip. There was a high degree of correlation between reactivity to the proteins produced by the two methods, (CSP (r=0.77; P<0.001), AMA1 (r=0.78; P<0.001). MSP2 (r=0.96; P<0.001)). To examine the reproducibility of the array probing, the reactivity against all HA spots from two independent chips was assessed and was highly correlated (r=0.92. P<0.001). Additionally, a smaller microarray chip was fabricated with a subset of 49 P. falciparum proteins and probed with the same plasma used to probe the larger 2320 fragment P. falciparum protein microarray. There was a strong correlation between plasma reactivity on the two microarrays (r=0.91, P<0.001).

In order to analyze low and high signal intensities (differential recognition) using standard statistical methods, the heteroskedastic nature of microarray platforms (Durbin, et al. Bioinformatics 18 Suppl. 1:S105-110 (2002): Ideker, et al., J. Comput. Biol 7:805-8) 7 (2000) and inherent variance-mean dependence in the data (Sundaresh, et al., Bioinformatics 22:1760-1766 (2006); Sundaresh, et al. Bioinformatics 23:1508-518 (2007)) needed to be considered and stabilized. Raw signal intensities were therefore variant log transformed by using either asinh (Excel 2007, Microsoft; (Sundaresh, et al., Bioinformatics 22:1760-1766 (2006)) or variance stabilizing normalization (vsn) (Bioconductor software (www.bioconductor.org) (Huber, et al. Bioinformatics 18 suppl. 1:S96-104 (2002)) transformation, to reduce the variance and experimental effects in the raw signal between differentially recognized antigens.

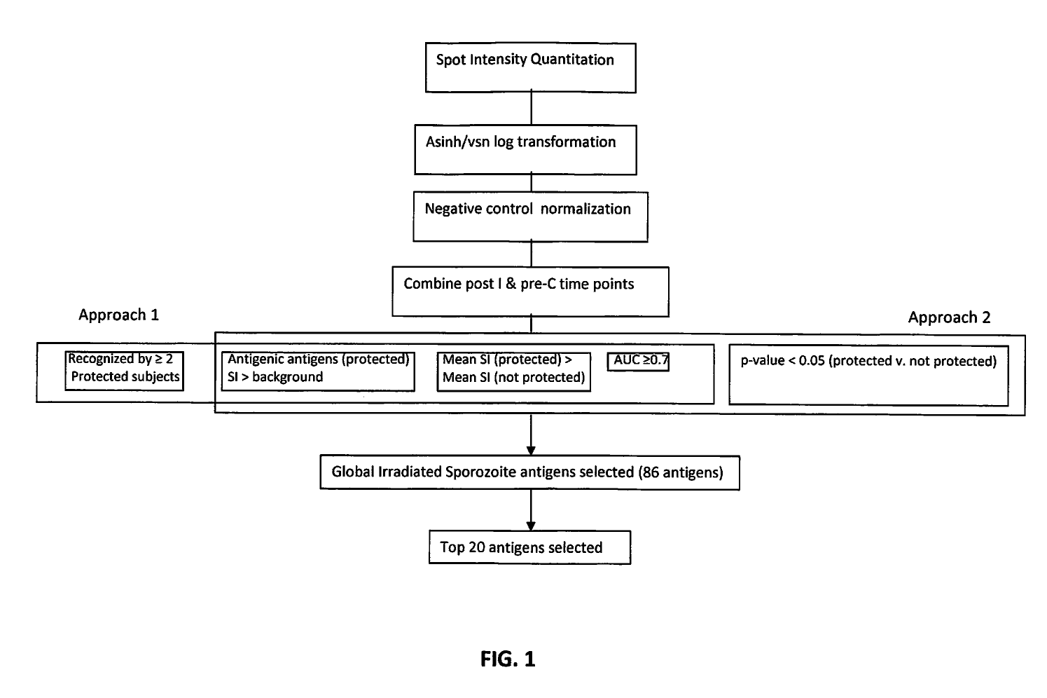

Before ranking and selection of antigens, data for each plasma sample was scaled by multiplication to a normalization ratio (average SI of all negative controls for that individual/average SI of all negative controls for all individuals) to apply the same baseline for all plasma. The post-immunization and pre-challenge time points for each individual in each group were combined for sample size purposes, since data showed no significant: differences between those time points, and data was biologically and statistically analyzed. Area under receiver operating characteristic curves (ROC) (AUC) and Bayesian regularized t-tests (R statistical environment software, www.rproject.org) were used to identify differentially recognized antigens between the protected and not protected groups (Baldi and Long, Bioinformatics 17:509-519 (2001)) (FIG. 1). Please note that the statistical testing (both AUC and p-values) have not under gone multiple testing correction, rather these statistics were used as a method to compare the two groups and rank the data accordingly. The identification of antigens of interest was made on the combination of the rankings in addition to other relevant criteria i.e. recognition in different groups.

The criterion for positive immunoreactivity to a given protein for both transformation methods was determined as an average signal intensity two standard deviation above the negative control (all individuals). Data were visually presented as a heatmap where colors represent the SI range for that antigen. Red represents high SI (reactivity), black represents intermediate reactivity and green represents low reactivity. Gene ontology (GO) annotation analysis was performed using the GOstats and org.Pf.plasmo.db R packages. The significance of annotation enrichment was assessed using Fisher's exact test.

A marked change in seroreactivity was noted at pre-challenge vs. post-challenge time points for the not protected individuals where there was a significant increase in overall signal intensity (SI) (P<2e-91) and number of antigens recognized (292 pre-challenge vs. 739 post-challenge), consistent with exposure to blood stage antigens upon development of patent parasitemia.

The profile for the infectivity control individuals also showed a significant increase in SI pre-challenge vs. post-challenge (P<2e-30) to a subset of antigens (289 prechallenge vs. 546 post-challenge) and 89% (546) of the antigens recognized post-challenge were also recognized by the not protected group post-challenge, and therefore appear to be expressed by the blood stage parasite. Minimal reactivity to P. falciparum antigens was noted with plasma from malaria naïve individuals or mock immunized individuals, consistent with a lack of exposure to P. falciparum.

To identify P. falciparum antigens putatively correlated with protection against sporozoite challenge, normalization and variance correction were performed using either the vsn and asinh methods. Since there were no significant changes in SI or number of antigens recognized at post-3^{rd} immunization or post-last immunization (prechallenge) time points for both protected and not protected groups [P<0.25 (not protected), P<0.2 (protected)], data from these biologically similar time points were combined for subsequent analyses. CyberT™ (Institute for Genomics and Bioinformatics, University of California, Irvine, Calif.) (a Bayesian regularized t-test) p-values and a receiver operating characteristic (ROC) curve of the mean SI were used to measure the power of an antigen to discriminate between protected and not protected cohorts, thus identifying antigens putatively associated with sporozoite-induced protection. The selection criteria are depicted in FIG. 1. An area under the ROC curve (AUC) value of 1.0 is a perfect prediction, 0.5 is a random chance, and <0.5 is a prediction in the wrong direction.

For both vsn and asinh approaches, antigens were selected according to the Following criteria: (i) antigenic (protected); i.e. average SI greater than two standard deviations (SD) above negative control; (ii) recognized by at least two protected individuals; (iii) average SI greater in the protected group than not protected group; (iv) an AUC value equal to or greater than 0.7; and (v) CyberT™ p-value <0.05 (protected vs. not protected).

Combining the antigens identified by either approach revealed a total of 86 fragments representing 77 proteins, termed “global IrrSpz” list. Five proteins (PF14_{—}0051, PFI0240c, PF10_{—}0183, PF10_{—}0211, MAL13P1.107) have two immunoreactive fragments on this list and two proteins (PF11_{—}0395, MAL7P1.146) have three fragments, since open reading frames (ORFs)>3000 bp were cloned as overlapping segments. When ranked by AUC values, five antigens on this list (6%) have an AUC value greater than 0.9 (1.5e-3<P>3.8e-3), and 31 antigens (36%) have an AUC greater than 0.8 (1.5e-3<P>4.43e-2).

The global IrrSpz list included three current vaccine candidates: AMA1 (PF11_{—}0344), CSP (PFC0210c), and SSP2/TRAP (PF13_{—}0201). Interestingly, these were highly antigenic in protected individuals, with 92% (AMA1), 100% (CSP) and 100% (SSP2/TRAP) recognition within this cohort, but were also recognized to a similar extent by not protected individuals (92%, 100%, 100%, respectively). The other 75 proteins on this list have not been previously characterized.

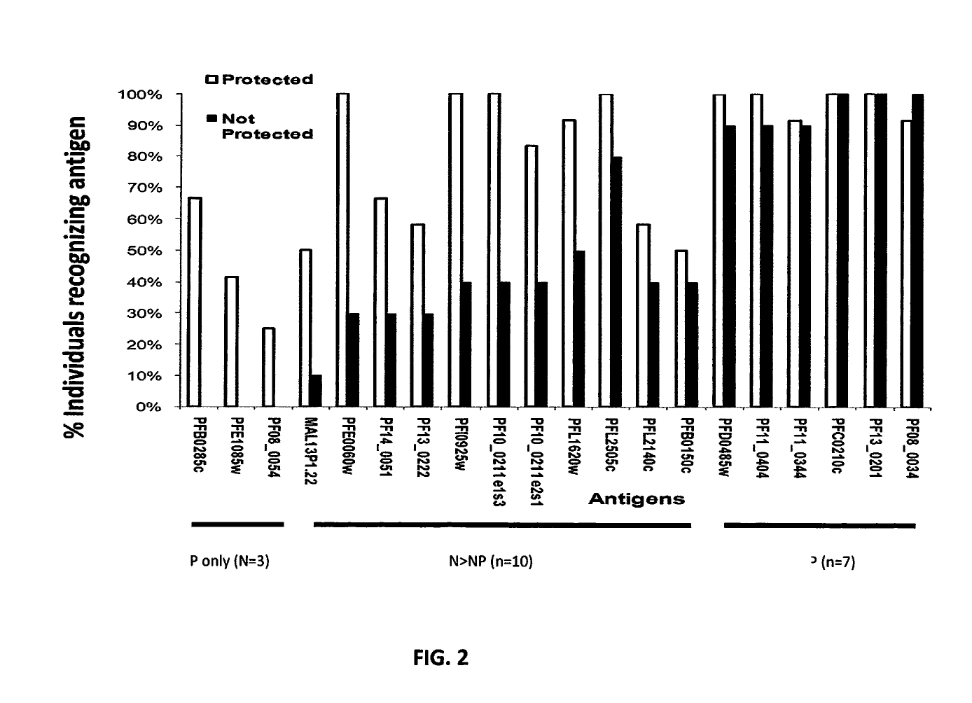

A subset of 20 fragments representing 19 proteins was common to both vsn and asinh approaches. These are shown in Table 1. Three of these antigens (i.e., PFB0285c, PFE1085w, PF08_{—}0054) were not recognized by not protected individuals and 10 of the antigens had a higher frequency of recognition in the protected cohort as compared with the not protected cohort. Seven antigens (including CSP, SSP2/TRAP, and AMA1) were recognized at a similar frequency by the two cohorts but the protected individuals had a significantly higher magnitude of response (0.0034<P>0.0077, 2-4 fold higher average SI) (FIG. 2).

TABLE 1
Characteristics of the Top 20 antigens highly associated with sporozoite-induced protection.

| | | | | | Average signal | | | | | |
|---|---|---|---|---|---|---|---|---|---|---|
| | | | | P value | intensity | | Fold enrich | | | |
| | | | | (Protected | | Not | (Protected | Frequency of recognition | | |
| | | | | v not | Pro- | Pro- | v not | Pro- | Not | Total # |
| Gene ID | Product Description | Details | AUC | Protected) | tected | tected | Protected) | tected | Protected | exons |
| PFI0925w | gamma-glutamylcysteine | exon 1 | 0.917 | 1.67E−03 | 3324 | 1293 | 3 | 100% | 40% | 1 |
| | synthetase | | | | | | | | | |
| PFB0285c | conserved Plasmodium | exon 1 | 0.917 | 2.77E−03 | 1341 | 337 | 4 | 67% | 0% | 1 |
| | protein, unknown | | | | | | | | | |
| | function | | | | | | | | | |
| PF14_0051 ¶ | DNA mismatch repair | exon 4 | 0.888 | 1.52E−03 | 2784 | 553 | 5 | 67% | 30% | 4 |
| | protein, putative | | | | | | | | | |
| PFD0485w | conserved Plasmodium | complete | 0.852 | 7.68E−03 | 12669 | 3205 | 4 | 100% | 90% | 1 |
| | protein, unknown | | | | | | | | | |
| | function | | | | | | | | | |
| PFL1620w | asparagine/aspartate rich | exon 1 | 0.833 | 1.06E−02 | 2471 | 807 | 3 | 92% | 50% | 3 |
| | protein, putative | | | | | | | | | |
| PF10_0211 § | conserved Plasmodium | exon 1 | 0.833 | 4.43E−02 | 1752 | 853 | 2 | 100% | 40% | 5 |
| | membrane protein, | | | | | | | | | |
| | unknown function | | | | | | | | | |
| PF10_0211 § | conserved Plasmodium | exon 2 | 0.833 | 4.43E−02 | 1752 | 853 | 2 | 83% | 40% | 5 |
| | membrane protein, | | | | | | | | | |
| | unknown function | | | | | | | | | |
| PFB0150c | protein kinase, putative | exon 2 | 0.824 | 2.37E−02 | 2528 | 620 | 4 | 50% | 40% | 1 |
| PF11_0344 | AMA1 | complete | 0.824 | 2.88E−02 | 4455 | 2248 | 2 | 92% | 90% | 1 |
| PFE0060w | PESP2 erythrocyte | exon 2 | 0.815 | 9.87E−03 | 5625 | 3030 | 2 | 100% | 30% | 2 |
| | surface protein | | | | | | | | | |
| PF08_0034 | histone acetyltransferase | exon 1 | 0.815 | 3.36E−02 | 2480 | 1293 | 2 | 92% | 100% | 4 |
| | GCN5, putative | | | | | | | | | |
| PF08_0054 | heat shock 70 kDa protein | complete | 0.806 | 1.52E−02 | 1214 | 221 | 5 | 25% | 0% | 1 |
| PFL2140c | ADP-ribosylation factor | exon 1 | 0.806 | 2.16E−02 | 3738 | 965 | 4 | 58% | 40% | 1 |
| | GTPase-activating protein | | | | | | | | | |
| PFC0210c | CSP | complete | 0.800 | 1.72E−02 | 15118 | 10066 | 2 | 100% | 100% | 1 |
| PFE1085w | DEAD-box subfamily | exon 1 | 0.796 | 1.03E−02 | 1201 | 203 | 6 | 42% | 0% | 1 |
| | ATP-dependent | | | | | | | | | |
| | helicase, putative | | | | | | | | | |
| PF11_0404 | transcription factor with | exon 2 | 0.787 | 3.42E−02 | 2907 | 1542 | 2 | 100% | 90% | 3 |
| | AP2 domain(s), putative | | | | | | | | | |
| PF13_0201 | SSP2/TRAP | complete | 0.769 | 3.11E−02 | 29578 | 9818 | 3 | 100% | 100% | 1 |
| PFL2505c | rhoptry neck protein 3, | exon 8 | 0.759 | 1.57E−02 | 4011 | 1531 | 3 | 100% | 80% | 8 |
| | putative | | | | | | | | | |
| PF13_0222 | phosphatase, putative | exon 1 | 0.750 | 4.98E−02 | 4243 | 960 | 4 | 58% | 30% | 1 |
| MAL13P122 | DNA ligase I | exon 2 | 0.713 | 3.91E−02 | 1739 | 466 | 4 | 50% | 10% | 2 |

| | | | | | | | | | Presence in | |
|---|---|---|---|---|---|---|---|---|---|---|
| | | | | | | | | | genomic/ | |
| | | | Mw | | # TM | # | Exported | Signal | proteomic | Functional categories |
| Gene ID | bp | aa | (kDa) | pl | domains | PEXEL/motif | protein | Peptide | datasets | (plasmoDB) |
| PFD825w | 3192 | 1063 | 124.46 | 5.1 | 0 | 0 | no | no | yes | Protein Synthesis |
| PFB0285c | 4311 | 1436 | 164.85 | 10.04 | 0 | 0 | no | no | yes | Hypothetical |
| PF14_0051 ¶ | 4548 | 1515 | 179.83 | 8.9 | 0 | 1 | no | yes | yes | Cell Cycle (DNA |
| | | | | | | | | | | processing) |
| PFD0485w | 1728 | 575 | 68.53 | 9.42 | 0 | 0 | no | no | yes | Hypothetical |
| PFL1620w | 16320 | 5439 | 646.34 | 6.38 | 0 | 1 | no | no | yes | Protein Synthesis |
| PF10_0211 § | 20805 | 6934 | 830.20 | 9.51 | 5 | 4 | no | no | yes | Hypothetical |
| PF10_0211 § | 20805 | 6934 | 830.20 | 9.51 | 5 | 4 | no | no | yes | Protein Synthesis |
| PFB0150c | 7458 | 2485 | 293.77 | 7.21 | 0 | 0 | no | no | yes | Hypothetical |
| PF11_0344 | 1869 | 622 | 72.04 | 5.23 | 1 | 0 | no | yes | yes | Cell Surface (Apical |
| | | | | | | | | | | organelle [symbol] |
| PFE0060w | 1227 | 408 | 48.72 | 7.19 | 3 | 1 | yes | yes | no | Hypothetical |
| PF08_0034 | 4398 | 1465 | 170.92 | 6.65 | 0 | 0 | no | no | yes | Metabolism |
| PF08_0054 | 2034 | 677 | 73.92 | 5.33 | 0 | 0 | no | no | yes | Cell Cycle (DNA |
| | | | | | | | | | | processing) |
| PFL2140c | 999 | 332 | 37.29 | 5.42 | 0 | 0 | no | no | yes | Hypothetical |
| PFC0210c | 1194 | 397 | 42.65 | 5.18 | 1 | 2 | no | yes | yes | Virulence |
| PFE1085w | 2526 | 841 | 97.34 | 7.96 | 0 | 3 | no | no | yes | Cell Cycle (DNA |
| | | | | | | | | | | processing) |
| PF11_0404 | 7962 | 2653 | 309.45 | 6.12 | 0 | 0 | no | no | yes | Transcription |
| PF13_0201 | 1725 | 574 | 64.74 | 4.7 | 0 | 1 | no | yes | yes | Hypothetical |
| PFL2505c | 6648 | 2215 | 263.16 | 9.62 | 3 | 0 | no | yes | yes | Hypothetical |
| PF13_0222 | 1728 | 575 | 67.56 | 5.08 | 0 | 0 | no | no | no | Protein Synthesis |
| MAL13P122 | 2739 | 912 | 104.51 | 7.66 | 0 | 1 | no | yes | yes | Cell Cycle (DNA |
| | | | | | | | | | | processing) |

[symbol]

indicates data missing or illegible when filed

The 10 antigens on the global list with the highest AUC values (0.86-0.93) also had very low p values (protected vs. not protected; 0.0015-0.033). Unexpectedly, all of these antigens had an average SI below 3000 (protected), suggesting that high antibody recognition and reactivity (i.e., serodominance) to a single antigen does not correlate with sporozoite-induced protection. Indeed, the average SI for 75 of the 86 antigens was below 5000, and 80 of the 86 were below 10000.

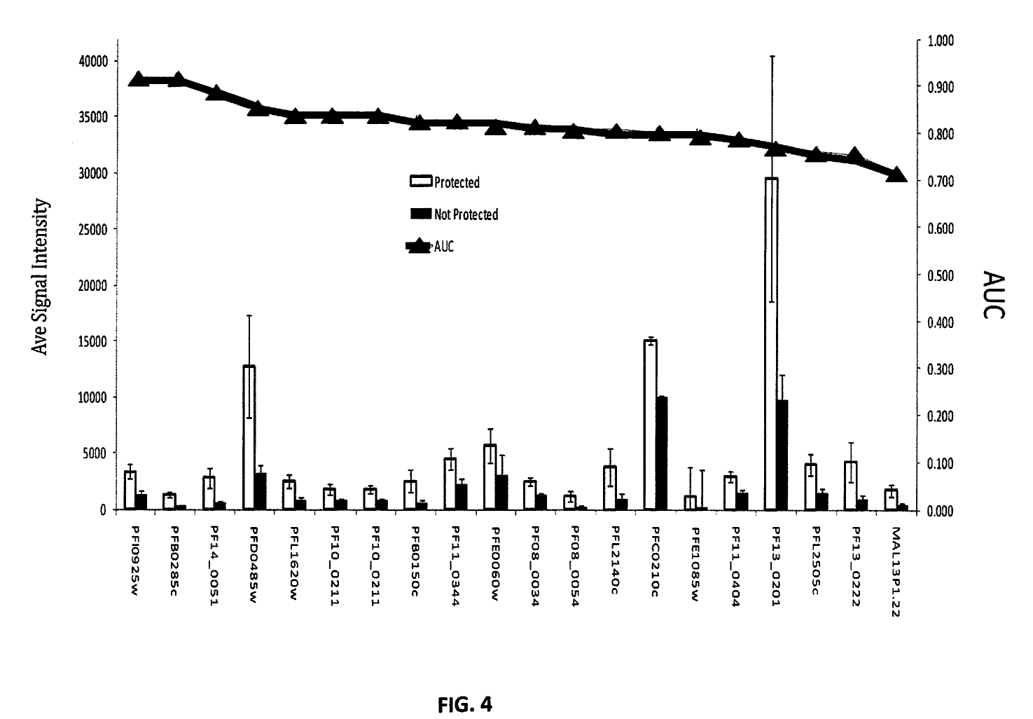

As shown in FIG. 2, protected individuals had a significantly greater cumulative antibody response to the antigens of Table 1, as compared to not protected individuals (P<0.0055). FIG. 4 shows the average signal intensities of the 20 antigens in Table 1 for each clinical group. This remarkable difference in cumulative response between the protected and non-protected individuals, suggests that sterile immunity is associated with a panel of antigens, not with individual antigens.

The majority of the global IrrSpz list (93%; 72/77 proteins) and genes in Table 1 (89%, 17/19 proteins) were detected via independent mass spectrometry (MS/MS) analysis of sporozoites (Florens, et al., Nature 419:520-526 (2002); Hall, et al., Science 307:82-86 (2005); Lasonder, et al., PLoS Pathog. 4:e1000195 (2008); Kappe, et al., Proc. Natl. Acad. Sci. (USA) 98:9895-9900 (2001)) or liver stage and/or sporozoite gene expression data (Kappe, et al., Proc. Natl. Acad. Sci. (USA) 98:9895-9900 (2001); Le Roch, et al., Science 301:1503-1508 (2003); Rosinski-Chupin, et al., BMC Genomics 8:466 (2007); Siau, et al., PLoS Pathog. 4:e1000121 (2008); Tarun, et al., Proc. Natl. Acad. Sci. (USA) 105:305-310 (2008)), PlasmoDB™), validating the expression of the genes during the pre-erythrocytic stage of the parasite life cycle.

### Example 2

### Characterization of Antigens Associated with Protection

This example illustrates the characterization of the proteins associated with protection. To identify specific genomic or proteomic features of antigens putatively associated with IrrSpz-induced protection, selected characteristics of the global IrrSpz list and the proteins listed in Table 1 were compared to the Plasmodium falciparum genome and to the known blood stage antigens (BSA) and sporozoite/liver stage antigens (SLA). The antigens from the global IrrSpz list and genes listed in Table 1 average respectively 3 and 2.5 fold larger for their transcription length, protein length and molecular weight as compared to the Plasmodium falciparum genome, BSA or SLA (Table 2); this is consistent with the fact that large proteins potentially present a greater number of B cell epitopes for antibody recognition. There was no difference in average isoelectric points (pI) or the number of exons compared to the P. falciparum genome, although the BSA and SLA had a lower average pI value than the other groups (Table 2).

TABLE 2
Comparison of genomic and proteomic features from antigens associated with
protection¹

| | | | Known | Known | |
|---|---|---|---|---|---|
| | Top 20 | Global | sporozoite/liver | blood stage | Pf |
| | list | list | stage antigens | antigens | genome |
| | (n = 20) | (n = 86) | (n = 7) | (n = 17) | (n = 5479) |
| Ave number of Exons | 2.3 | 3 | 1.6 | 1.9 | 2.5 |
| Ave Transcription Length (bp) | 5711 | 6909 | 2245 | 2274 | 2270 |
| Ave protein length (aa) | 1902 | 2302 | 747 | 760 | 755 |
| Ave pI value | 7.1 | 7.8 | 5.3 | 5.3 | 8 |
| Ave Mw (kDa) | 224 | 272 | 85 | 87 | 89 |
| Number with TM domains | 30% | 34% | 71% | 41% | 31% |
| Number with Signal peptide | 35% | 24% | 100% | 65% | 19% |
| Number with PEXEL motif | 45% | 53% | 71% | 23% | 27% |

¹Characteristics of the global irradiated sporozoite list and top 20 list (i.e., antigens in Table 1) are compared to known vaccine candidates and the P. falciparum genome (PlasmoDB ™). Attributes such as average (Ave) number of exons, gene (bp) and protein (aa) size, isoelectric point (pI), presence of features that are recognized by antibodies (transmembrane domains, signal peptide, (PEXEL motif), were compared.

An enrichment of Plasmodium export element (PEXEL) motifs was noted in a global IrrSpz list (2-fold, Table 2) compared to whole genome and known BSA. The global list of revealed 86 fragments representing 77 proteins.

PEXEL motifs are found in five of seven known SLA, consistent with a function in the transport of liver stage parasite proteins to hepatocytes. Although signal peptides have been identified in all known SLA and 11 of 17 known BSA, consistent with a role for secretion in inducing antibody responses, antigens containing a signal peptide were not enriched in the proteins in Table 1 (35%, 7/20, p-value=0.08) or global list (24%, 21/68, p-value=0.31). These data are consistent with the concept that IrrSpz protection is mediated by T cells rather than antibodies. Also consistent with this is the observation that only 30% (p-value=0.64) and 34% (p-value=0.9) of genes in Table 1 or Global IrrSpz list antigens, respectively, had defined transmembrane domains, which are associated with exposure of surfaces for antibody recognition (Table 2).

Identified proteins were assigned functional categories based on annotation information available from PlasmoDB (PlasmoDB: a functional genomic database for malaria parasites. Nucleic Acids Res. 2008 Oct. 31. Aurrecoechea C, et al). Fifty-six percent of the global and 40% of the 20 sterile immunity-associated proteins were hypothetical proteins, with the remainder assigned to multiple functional categories. Interestingly, the functional categories for the proteins in Table 1 showed a notable concentration of genes/proteins involved in or regulating DNA processing/cell cycle (20%) and protein synthesis (20%): cellular communication, transport facilitation and protein fate were not represented in this subset. Gene ontology analysis also indicates an overrepresentation of biological processes involving DNA, invasion and metabolic processes, DNA related activity and perhaps a localization of these proteins for invasion purposes.

### Example 3

### Immunogenic Composition and Method of Use for Immunizing Against Malaria Using Identified Proteins

This example illustrates a method of inducing an immune response against malaria. In a preferred embodiment, one or more of the identified proteins listed in Table 1, associated with sterile immunity, can be incorporated in an immunogenic composition, such as a vaccine. In one example, all of the proteins listed in Table 1 can be incorporated into an immunogenic composition.

In this embodiment, the immunogenic composition can be a subunit immunogenic composition or vaccine, composed of one or more of the isolated proteins, or immunogenic fragments or derivatives of the proteins, selected from the list of proteins in Table 1 that afforded sterile protection against malaria. Alternatively, an immunogenic composition or vaccine can be composed of nucleic acid, encoding one or more of the proteins of Table 1, or immunogenic fragments or derivatives of the proteins, inserted into one or more expression systems suitable for expression in mammals, such as humans. Table 3 lists the proteins from Table 1 and their associated sequence identification numbers.

TABLE 3
Summary of Sequence Numbers

| Protein | Sequence ID | SEQ ID. No. |
|---|---|---|
| Designation | No. (DNA) | (Polypeptide) |
| PF10925w | 1 | 2 |
| PFB0285c | 3 | 4 |
| PF14_0051 | 5 | 6 |
| PFD0485w | 7 | 8 |
| PFL1620w | 9 | 10 |
| PF10_0211 | 11 | 12 |
| PFB0150c | 13 | 14 |
| PF11_0344 | 15 | 16 |
| PFE0060w | 17 | 18 |
| PF08_0034 | 19 | 20 |
| PF08_0054 | 21 | 22 |
| PFL2140c | 23 | 24 |
| PFC0210c | 25 | 26 |
| PFE1085w | 27 | 28 |
| PF11_0404 | 29 | 30 |
| PF13_0201 | 31 | 32 |
| PFL2505c | 33 | 34 |
| PF13_0222 | 35 | 36 |
| MAL13P1.22 | 37 | 38 |

It is contemplated that the antigen(s) can be expressed either as a component of a DNA vaccine or other platform system. An example of a contemplated expression system includes, but is not limited to, viral systems, including replicating and nonreplicating vectors. Examples of contemplated viral vectors include adenovirus, alphavirus, poxvirus, cytopmegalovirus, canine distemper virus and yellow fever virus. The antigen(s) could be incorporated as an insert of a DNA or other vaccine expression system, either as a single antigen or multiple antigen expression systems from a single or multiple promoters.

The contemplated invention includes a method for inducing an immune response in mammals, including humans. In this example, antigen(s), either as polypeptide or incorporated into a nucleic acid expression system, such as a DNA or viral system, are administered in one or more doses. The method also contemplates inducing an immune response utilizing a prime-boost immunization regimen. In this embodiment, one or more priming immunization doses would be administered followed by one or more boosting immunizations.

The priming and boosting immunization comprises a composition containing a malaria polypeptide, wherein the polypeptide contains one or more of the polypeptide sequences of Table 3, or immunogenic derivatives, thereof. Alternatively, the immunogenic composition can comprise an expression system capable of expressing the polypeptides in mammals. In this embodiment, nucleic acid molecules, listed in Table 3 or encoding the polypeptides, or derivatives thereof, of Table 3 can be inserted into a DNA plasmid or a viral expression vectors. Examples of viral expression vector systems include: alphavirus (and alphavirus replicons), adenovirus, poxvirus, adeno-associated virus, cytomegalovirus, canine distemper virus, yellow fever virus and retrovirus.

The contemplated methods also include immunization regimens wherein the priming immunization comprises malarial peptides expressed from a DNA plasmid expression vector or an adenovirus, while the boosting immunization includes malaria peptides expressed from either: adenovirus, adenovirus that is heterologous to the priming adenovirus, poxvirus or one or more malaria polypeptides. The expressed malaria polypeptide and encoding nucleic acid can be any of a number of malarial polypeptides and nucleic acid sequences Table 3, or immunogenic derivatives of the polypeptides, thereof.

### Example 4

### Blood Stage Specific Antigens

A strong immune response to all well characterized blood stage antigens was also seen post-challenge in a cohort that was not afford protection. As expected, negligible responses to antigens known to be expressed at the blood stage were observed in the protected cohort and no changes in their heatmap profile occurred throughout the study time course.

Analysis of the responses post challenge for the not protected cohort allowed identification of novel blood stage specific P. falciparum antigens, using the following criteria: (i) antigenic; i.e. average SI greater than two SDs above negative control in at least two not protected individuals; (ii) statistically significant recognition (p) after challenge as compared to pre challenge <0.05 (not protected); and (iii) not recognized by protected subjects.

As illustrated in Table 4, a total of 23 antigens met these criteria, of which two have been previously characterized (MSP1 [PFI1475w] and liver stage antigen 3 [LSA3, PFB0915w]) and 21 are novel. The sequence identification numbers of the proteins in Table 4 and the DNA encoding these proteins are shown in Table 5.

TABLE 4

| | | | Present in | | Irradiated | |
|---|---|---|---|---|---|---|
| | | | orthologue | Naturally | sporozoite and | |
| | Present | Present | P. yoelii | acquired | naturally acquired | |
| | iRBC MS | Merozoite MS | blood stage | immunity | immunity study | |
| | (Florens, | (Florens, | (Watanabe, | (Crompton, | (Doolan, | |
| Gene ID | et al., (2004)) | et al., (2002)) | et al., (2007)) | et al., (2010)) | et al., (2008)) | Protein description |
| PFI0855w | No | No | No | Yes | No | Hypo. Protein |
| PF14_0198 | Yes | Yes | Yes | No | No | Gly. tRNA ligase |
| | | | | | | (putative) |
| PFA0535c | No | No | Yes | No | No | Kinesin (putative) |
| PF08_0018 | No | No | Yes | No | No | Translation initiation |
| | | | | | | factor-like protein |
| PFL1705w | No | No | Yes | No | No | RNA binding protein |
| | | | | | | (putative) |
| MAL6P1.146 | No | No | No | No | No | P. falciparum PK4 |
| | | | | | | protein kinase |
| PFL0410w | No | YES | YES | NO | NO | Cys. Repeat modular |
| | | | | | | protein 3 |
| PFL2440w | No | No | Yes | No | No | DNA repair protein rhp |
| | | | | | | 16 (putative) |
| PFL2520w | No | No | Yes | No | No | Reticulocyte binding |
| | | | | | | protein (pseudogene) |
| PF14_0084 | No | Yes | Yes | No | No | Hypothetical protein |
| PFI1500w | No | No | Yes | No | No | Hypothetical protein |
| PF14_0327 | No | No | Yes | No | No | Methionine |
| | | | | | | aminopeptidase, type II |
| PF11_0270 | Yes | Yes | No | Yes | No | Threonine-tRNA ligase |
| | | | | | | (putative) |
| PF11_0479 | No | Yes | Yes | No | No | Hypothetical |
| PFI0755c | Yes | Yes | Yes | No | No | 6-phosphofructokinase |
| | | | | | | (putative) |
| PF11_0091 | No | Yes | Yes | No | No | Transcription factor |
| | | | | | | (putative) |
| PF14_0315 | No | Yes | Yes | No | No | Hypothetical protein |
| MAL7P1.137 | No | No | Yes | No | No | Kelch protein (putative) |
| PFI1510w | No | No | Yes | No | No | Nucleolar protein Nop 52 |
| | | | | | | (putative) |
| PFI1475w | Yes | Yes | Yes | No | Yes | MSP1 |
| PFB0915w | No | Yes | No | No | Yes | LSA3 |
| PF10_0323 | Yes | Yes | No | No | No | Hypothetical protein |
| MAL8P1.23 | No | Yes | Yes | No | No | Ubiquitin-protein ligase |
| | | | | | | 1 (putative) |

TABLE 5

| | SEQ ID No. |
|---|---|
| Gene ID | (DNA/Protein) |
| PFI0855 | 39/40 |
| PF14_0198 | 41/42 |
| PFA0535c | 43/44 |
| PF08_0018 | 45/46 |
| PFL1705w | 47/48 |
| MAL6P1.146 | 49/50 |
| PFL0410w | 51/52 |
| PFL2440w | 53/54 |
| PFL 2520w | 55/56 |
| PF14_0084 | 57/58 |
| PFI1500w | 59/60 |
| PF14_0327 | 61/62 |
| PF11_0270 | 63/64 |
| PF11_0479 | 65/66 |
| PFI0755c | 67/68 |
| PF11_0091 | 69/70 |
| PF14_0315 | 71/72 |
| MAL7P1.137 | 73/74 |
| PFI1510w | 75/76 |
| PFI1475w | 77/78 |
| PFB0915w | 79/80 |
| PF10_0323 | 81/82 |
| MAL8P1.23 | 83/84 |

For 22 of the 23 antigens, evidence of their expression at the blood stage has been independently reported from data sets of P. falciparum infected red blood cell MS/MS (Florens, et al., Mol. Biochem. Parasitol. 135:1-11 (2004)), P. falciparum merozoite MS/MS (Florens, et al., Nature 419:520-526 (2002)), P. yoelii 17XNL asexual blood stage EST datasets (P. falciparum orthologs) (Watanabe, et al., Nucleic Acids Res. 35:D431-438 (2007)) and previous P. falciparum protein microarrays which analyzed plasma from individuals with blood stage malaria (Crompton, et al., PNAS 107:6958-6963 (2010); Doolan, et al., Proteomics, 8:4680-4694 (2008)). Four antigens, expressed in both the blood stage and the sporozoite/liver stage of the parasite life cycle, were also identified.

In a preferred embodiment, one or more of the proteins of Table 4, the sequence identification numbers of which are shown in Table 5, or immunogenic fragments or derivatives of these proteins can be incorporated into an immunogenic composition. Alternatively, the immunogenic composition can comprise DNA encoding the proteins, fragments or derivatives. In this embodiment, DNA encoding the proteins of Table 4, the sequence identification numbers of which are shown in Table 5, can be in one or more expression vectors capable of expression in mammalian hosts. As a further embodiment, one or more of the proteins of Table 4, immunogenic fragments or derivatives, or DNA encoding these proteins, can be incorporated into immunogenic an immunogenic composition with one or more of the proteins of Table 3, or immunogenic fragments or derivatives of these proteins, or DNA encoding these proteins.

It is contemplated that the antigens can be expressed either as a component of a DNA vaccine, or other platform system. An example of a contemplated expression system includes, but is not limited to, viral systems, including replicating and nonreplicating vectors. Examples of contemplated viral vectors include adenovirus, alphavirus, poxvirus, cytopmegalovirus, canine distemper virus and yellow fever virus. The antigen could be incorporated as an insert of a DNA or other vaccine expression system, either as a single antigen or multiple antigen expression systems from a single or multiple promoters.

Another embodiment is a method for inducing an immune response in mammals, including humans utilizing the expressed proteins listed in Table 4. The sequence listings corresponding to the DNA sequences of the genes, and their protein expression products, are listed in Table 5. In this embodiment, antigens, either as polypeptide or incorporated into a nucleic acid expression system, such as a DNA or viral system, are administered in one or more doses. The method also contemplates inducing an immune response utilizing a prime-boost immunization regimen. In this embodiment, one or more priming immunization doses would be administered followed by one or more boosting immunizations.

The priming and boosting immunization comprises a composition containing a malaria polypeptide, wherein the polypeptide contains one or more of the polypeptide sequences of Table 5, or immunogenic derivatives, thereof. Alternatively, the immunogenic composition can comprise an expression system capable of expressing the polypeptides in mammals. In this embodiment, nucleic acid molecules, listed in Table 5 or encoding the polypeptides, or derivatives thereof, of Table Scan be inserted into a DNA plasmid or a viral expression vectors. Examples of viral expression vector systems include: alphavirus (and alphavirus replicons), adenovirus, poxvirus, adeno-associated virus, cytomegalovirus, canine distemper virus, yellow fever virus and retrovirus.

The contemplated methods also include immunization regimens wherein the priming immunization comprises malarial peptides expressed from a DNA plasmid expression vector or an adenovirus, while the boosting immunization includes malaria peptides expressed from either: adenovirus, adenovirus that is heterologous to the priming adenovirus, poxvirus or one or more malaria polypeptides. The expressed malaria polypeptide and encoding nucleic acid can be any of a number of malarial polypeptides and nucleic acid sequences shown in Table 5, or immunogenic derivatives of the polypeptides, thereof.

All references, including publications, patent applications and patents, cited are herein incorporated by reference.

Having described the invention, one of skill in the art will appreciate in the appended claims that many modifications and variations of the present invention are possible in light of the above teachings. It is therefore, to be understood that, within the scope of the appended claims, the invention may be practiced otherwise than as specifically described.

What is claimed is:

1. An immunogenic composition comprising isolated proteins, or immunogenic fragments or derivatives, thereof, wherein said proteins are associated with sterile immunity against malaria.

2. The immunogenic composition of claim 1, wherein the composition comprises one or more of said the isolated proteins, immunogenic fragments or derivatives, wherein said isolated proteins are associated with the loci selected from the group consisting of PF10925w; PFB0285c; PF14_{—}0051; PFD0485w; PFL1620w; PF10_{—}0211; PFB0150c; PFE0060w; PF08_{—}0034; PF08_{—}0054; PFL2140c; PFC0210c; PFE1085w; PF11_{—}0404; PFL2505c; PF13_{—}0222; and MAL13P1.22 or immunogenic fragments or derivatives, thereof.

3. The immunogenic composition of claim 2, wherein the composition also comprises one or both of the isolated proteins, immunogenic fragments or derivatives associated with the loci PF11_{—}0344 and PFf13_{—}0201 or immunogenic fragments or derivatives, thereof.

4. The immunogenic composition of claim 2, wherein said isolated proteins have the amino acid sequences selected from the group consisting of SEQ ID NOs: 2, 4, 6, 8, 10, 12, 14, 18, 20, 22, 24, 26, 28, 30, 34, 36 and 38, or immunogenic fragments or derivatives, thereof.

5. The immunogenic composition of claim 4, wherein said composition also comprises one or both of the proteins with amino acid sequences of SEQ ID NOs.: 16 and 32, or immunogenic fragments or derivatives, thereof.

6. An immunogenic composition comprising one or more isolated nucleic acid molecules encoding one or more proteins, or immunogenic fragments or derivatives, wherein said proteins are associated with sterile immunity against malaria, wherein said isolated nucleic acid molecules are expressed in an expression vector capable of expression in a mammal.

7. The immunogenic composition of claim 6, wherein said nucleic acid molecules are encoded by the nucleic acid sequences selected from the group consisting of SEQ ID NOs: 1, 3, 5, 7, 9, 11, 13, 17, 19, 21, 23, 25, 27, 29, 33, 35, and 37 or a substantial portion or ortholog, thereof.

8. The immunogenic composition of claim 6, wherein said composition also comprises one or both of the DNA sequences 15 and 31 or a substantial portion or ortholog, thereof.

9. A method of inducing an immune response against malaria in a mammal, which method comprises administering to a mammal a composition comprising one or more isolated nucleic acid molecules, inserted into a suitable expression vector, encoding isolated malaria polypeptides, wherein said isolated nucleic acid molecules are as in claim 4 or 5.

10. The method of claim 9, where the suitable expression system is a DNA plasmid or replicating or nonreplicating viral vector.

11. The method of claim 9, wherein said method further comprises one or more priming and one or more boosting immunizations, wherein said priming immunizations comprise one or more malaria polypeptides, as in claim 2 or 3, or comprise a composition comprising one or more said isolated nucleic acid molecules, as in claim 7 or 8, inserted into a suitable expression vector, and wherein said boosting immunizations comprise a malaria polypeptide, as in claim 2 or 3, or comprise a composition comprising one or more isolated nucleic acid molecules, as in claim 7 or 8, inserted into suitable expression vector.

12. The method of claim 9, wherein said suitable expression vector is selected from the group consisting of DNA plasmid alphavirus replicon, adenovirus, poxvirus, adenoassociated virus, cytomegalovirus, canine distemper virus, yellow fever virus and retrovirus.

13. The method of claim 11, wherein said suitable expression vector is selected from the group consisting of DNA plasmid, alphavirus replicon, adenovirus, poxvirus, adenoassociated virus, cytomegalovirus, canine distemper virus, yellow fever virus and retrovirus.

14. The method of 11, wherein said priming immunization vector is an alphavirus vector and said boosting immunization vector is nonalphavirus vector.

15. The method of claim 11, wherein said priming immunization comprises an expression vector that is a DNA plasmid or an adenovirus and the boosting immunization is selected from the group consisting of adenovirus, adenovirus heterologous to the priming adenovirus, poxvirus and polypeptide, wherein said polypeptides have amino acid sequences as in claim 4.

16. The method of claim 12, wherein the alphavirus replicon preparation is selected from the group consisting of RNA replicons, DNA replicons and alphavirus replicon particles.

17. The method of claim 12, wherein the alphavirus is selected from the group consisting of Venzuelean Equine Encephalitis Virus, Semliki Forest Virus and Sindbis Virus.

18. The method of claim 14, wherein the non-alphavirus viral expression system is selected from the group consisting of poxvirus, adenovirus, adeno-associated virus and retrovirus.

19. The method of claim 15, wherein the poxvirus is selected from the group consisting of cowpox, canarypox, vaccinia, modified vaccinia Ankara, or fowlpox.
